SEQUENCE LISTING (14 sequences)

SEQ ID NO: 1 - protein, 108 aa, Artificial Sequence [sequence omitted]

SEQ ID NO: 2 - DNA, 324 bp, Artificial Sequence
  Synthetic Construct
gacatccagatgacccagtctccatcctccctgtctgcatctgtaggagacagagtcaccatcacttgcagtgtcagctcaagtgtatcctccatttacttgcactggtatcagcagaaaccagggaaagcccctaagctcctgatctatagcacatccaacttggcttctggagtcccatcaaggttcagtggcagtggatctgggacagatttcactctcaccatcagcagtctgcaacctgaagattttgcaacttactactgtcaagtctacagtggttacccgctcacgttcggcggagggaccaaggtggagatcaaa

SEQ ID NO: 3 - protein, 115 aa, Artificial Sequence [sequence omitted]

SEQ ID NO: 4 - DNA, 342 bp, Artificial Sequence
  Synthetic Construct
caggttcagctggtgcagtctggtgctgaggtgaagaagcctggtgcctcagtgaaggtctcctgcaaggcttctggttacacctttaccgactactacatgcactgggtgcgtcaggcccctggtcaaggtcttgagtggatgggtcgtgttaatcctaaccggaggggtactacctacaaccagaaattcgagggccgtgtcaccatgaccacagacacatccacgagcacagcctacatggagctgcgtagcctgcgttctgacgacacggccgtgtattactgtgcgcgtgcgaactggcttgactactggggccagggcaccaccgtcaccgtctcc

SEQ ID NO: 5 - protein, 215 aa, Artificial Sequence
  Synthetic Construct
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 645 bp, Artificial Sequence
  Synthetic Construct
gacatccagatgacccagtctccatcctccctgtctgcatctgtaggagacagagtcaccatcacttgcagtgtcagctcaagtgtatcctccatttacttgcactggtatcagcagaaaccagggaaagcccctaagctcctgatctatagcacatccaacttggcttctggagtcccatcaaggttcagtggcagtggatctgggacagatttcactctcaccatcagcagtctgcaacctgaagattttgcaacttactactgtcaagtctacagtggttacccgctcacgttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 7 - protein, 441 aa, Artificial Sequence
  Synthetic Construct
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 8 - DNA, 1323 bp, Artificial Sequence
  Synthetic Construct
caggttcagctggtgcagtctggtgctgaggtgaagaagcctggtgcctcagtgaaggtctcctgcaaggcttctggttacacattcactgactactacatgcactgggtgcgtcaggcccctggtcaaggtcttgagtggatgggtcgtgttaatcctaaccggaggggtactacctacaaccagaaattcgagggccgtgtcaccatgaccacagacacatccacgagcacagcctacatggagctgcgtagcctgcgttctgacgacacggccgtgtattactgtgcgcgtgcgaactggcttgactactggggccagggcaccaccgtcaccgtctcctccgcctccaccaagggcccatcggtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 9 - protein, 932 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFT

SEQ ID NO: 10 - protein, 908 aa, Homo sapiens
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFT

SEQ ID NO: 11 - protein, 283 aa, Homo sapiens
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKR

SEQ ID NO: 12 - protein, 212 aa, Homo sapiens
STKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLG

SEQ ID NO: 13 - protein, 214 aa, Artificial Sequence
  Synthetic Construct
EIQMTQSPASLSASVGETVTITCRASENIYSYLAWYQRKQGRSPQLLVYNAKPLAEGVPSRFSGSGSGTQFSLKINSLQPEDFGTYYCQHHYGTPFTFGSGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 443 aa, Artificial Sequence
  Synthetic Construct
EVQLQQSGTVLARPGASVKMSCKASGYSFTSYWMYWVKQRPGQGLEWIGGFHPRNSGTNYNQKFKGKAKLTAVTSASTAYMELSSLTNEDSAVYYCTRGYYYDGSFTYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

(19) United States
(12) Patent Application Publication
(10) Pub. No.: US 2021/0283249 A1
(43) Pub. Date: Sep. 16, 2021

(54) COMBINATION THERAPY FOR CANCER WITH EXON 14 SKIPPING MUTATION(S) OR EXON 14 SKIPPING PHENOTYPE

(71) Applicant: Eli Lilly and Company, Indianapolis, IN (US)

(72) Inventor: Sau-Chi Betty YAN, Indianapolis, IN (US)

(21) Appl. No.: 16/345,992

(22) PCT Filed: Nov. 10, 2017
(86) PCT No.: PCT/US2017/061028

§ 371 Date: Apr. 29, 2019

Related U.S. Application Data
(60) Provisional application No. 62/422,887, filed on Nov. 16, 2016.
(60) Provisional application No. 62/555,779, filed on Sep. 8, 2017.

(51) Int. Cl. A61K 39/395; A61P 35/00; A61K 31/4439; A61K 9/00

(52) U.S. Cl. CPC A61K 39/3955; A61P 35/00; A61K 2039/545; A61K 9/0053; A61K 9/0019; A61K 31/4439

(57) ABSTRACT

The present invention provides preparation of medicaments for use in treating and methods of treating cancer with tumors with MET exon 14 skipping mutation(s) or MET exon 14 skipping phenotype with emibetuzumab, in combination, as described herein, with merestinib.

The present invention relates to combinations of a ligand neutralizing and MET receptor internalizing bivalent anti-human MET antibody, preferably, emibetuzumab, with an anti-human MET kinase inhibitor, preferably, merestinib, or a pharmaceutically acceptable salt thereof, and to methods of using the combinations to treat certain disorders, such as lung cancer and gastric cancer, in patients with tumors bearing MET exon 14 skipping(s) or MET exon 14 skipping phenotype.

Overexpression and activation of MET tyrosine receptor kinase can be an oncogenic driver of tumor growth in many types of cancer. The MET signaling pathway regulates a wide variety of normal cellular functions that can be subverted to support neoplasia, including cell proliferation, survival, apoptosis, scattering and motility, invasion, and angiogenesis. MET over-expression (with or without gene amplification), aberrant autocrine or paracrine ligand production, and missense MET mutations are mechanisms that lead to activation of the MET pathway in tumors and are associated with poor prognostic outcome. Combinations of MET inhibitors and MET antibodies have been contemplated in the art. See, US 20140037625 and WO 2012031027.

Different genomic changes may occur in the intronic and/or exonic segments of MET and can lead to an alternatively spliced transcript of MET where exon 14 is skipped (i.e., exon 14 is largely or entirely deleted). MET exon 14 skipping mutations result in a protein missing the Y1003 phosphorylation site, the binding site for the ubiquitin ligase CBL, which targets MET for degradation. Additionally, a single point mutation at Y1003, D1002 or R1004 will also result in an inability of the ubiquitin ligase CBL to bind to the MET receptor without skipping of exon 14 (i.e., MET exon 14 skipping phenotype). This results in a MET protein with increased stability and oncogenic potential. MET exon 14 skipping mutations or an exon 14 skipping phenotype has been observed in adenosquamous, adenocarcinoma, sarcomatoid, squamous cell, large cell, and small cell histologies. Specifically, MET exon 14 skipping has been detected in lung adeonocarcinoma, as well as in neuroblastoma, gastric, and colon cancer cell lines. Tumors with MET exon 14 skipping mutations have been reported to be responsive to treatment with MET inhibitors in clinical case reports and series.

MET exon 14 skipping mutation (s) or exon 14 skipping phenotype are targetable mutations in lung cancer and is reported in approximately 3 to 6% of non-small cell lung cancer (NSCLC) patients. Lung cancer remains the third most prevalent cancer in the United States and is the leading cause of cancer death in both men and women throughout the world. The two main types of lung cancer are small cell lung cancer and NSCLC. The majority of patients with lung cancer have advanced and/or metastatic disease at diagnosis and the majority of patients treated with curative intent develop recurrence. These patients present with advanced, inoperable stage cancer for which there is no prospect of cure. Treatment is provided to improve symptoms, optimize quality of life, and prolong survival.

MET exon 14 skipping mutation(s) or MET exon 14 skipping phenotype are also targetable mutations in gastric cancer, a malignant tumor that originates in the stomach lining. Gastric cancers are classified according to the type of tissue from which they originate, with the most common type being adenocarcinoma and accounts for over 90% of all stomach cancers. Adenocarcinoma of the esophagus including carcinoma of the gastroesophageal junction (GEJ) is one of the fastest rising malignancies and is associated with a poor prognosis. Other forms of gastric cancer include lymphomas and sarcomas. Gastric cancer may be cured if it is found and treated at an early stage, but unfortunately, it is often found at a later stage.

There remains a need for the treatment of cancers with tumors bearing MET exon 14 skipping mutation(s) or MET exon 14 skipping phenotype. Tumors bearing MET exon 14 skipping mutation(s) or MET exon 14 skipping phenotype can have a response to MET inhibitors. Thus, a combination of an anti-human MET inhibitor, preferably, merestinib, or a pharmaceutically acceptable salt thereof, with an anti-human MET neutralizing and internalizing bivalent antibody, preferably, emibetuzumab, may provide a treatment option for cancer patients who have tumors bearing MET exon 14 skipping mutations or MET exon 14 skipping phenotype.

N-(3-Fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide (CAS #1206799-15-6), also known as merestinib and represented by the structural formula (I) below, is a small molecule type II MET kinase inhibitor. Merestinib and methods of making and using this compound and pharmaceutically acceptable salts thereof including for the treatment of neoplastic diseases such as solid and non-solid tumors are disclosed in WO 2010/011538. Furthermore, merestinib is currently being evaluated in Phase 2 clinical studies for patients in NSCLC and solid tumors (see ClinicalTrials.gov NCT02920996).

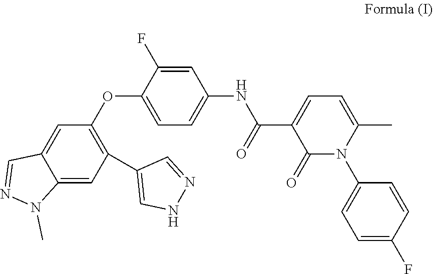 [SMILES: Cc1ccc(C(=O)Nc2ccc(Oc3cc4cnn(C)c4cc3-c3cn[nH]c3)c(F)c2)c(=O)n1-c1ccc(F)cc1]

Anti-human MET monoclonal antibodies, including, but not limited to, C8-H241 antibodies, were previously disclosed in WO 2010/059654. Emibetuzumab is a C8-H241-IgG4 antibody comprising: two light chains, each of whose amino acid sequence is that given in SEQ ID NO: 5, and two heavy chains, each of whose amino acid sequence is that given in SEQ ID NO: 7 (see, WHO Drug Information, Proposed International Nonproprietary Names (INN) List 111, Volume 28, No. 2, July 2014). Emibetuzumab has high neutralization and internalization activities resulting in inhibition of both HGF-dependent and HGF-independent (e.g., MET amplified) MET pathway activation and tumor growth in xenograft models. Emibetuzumab is currently being evaluated in Phase 2 clinical studies in combination with erlotinib in NSCLC patients (see, ClinicalTrials.gov NCT01900652, NCT01897480).

The present invention provides methods of treating cancer with tumors bearing MET exon 14 skipping mutation(s) or MET exon 14 skipping phenotype by using a novel combination of emibetuzumab and merestinib, or a pharmaceutically acceptable salt thereof, as part of a specific treatment regimen that provides unexpected beneficial therapeutic effects from the combined activity of these therapeutic agents in some cancer patients as compared to the therapeutic effects provided by either agent alone. Preferably, the cancer is lung, neuroblastoma, gastric, or colon cancer. More preferably, the cancer is gastric or lung cancer. Even more preferably, the cancer is lung cancer. Most preferably, the cancer is non-small cell lung cancer.

Accordingly, the present invention provides a method of treating cancer with tumors bearing MET exon 14 skipping mutation(s) or MET exon 14 skipping phenotype in a patient, comprising administering to a cancer patient in need of such treatment an effective amount of emibetuzumab in combination with an effective amount of an anti-MET inhibitor. In a further embodiment, the anti-MET inhibitor is merestinib, or a pharmaceutically acceptable salt thereof. Preferably, the cancer in the aforementioned methods is lung, neuroblastoma, gastric, or colon cancer. More preferably, the cancer is gastric or lung cancer. Even more preferably, the cancer is carcinoma of the gastroesophageal junction. Even more preferably, the cancer is lung cancer. Most preferably, the cancer in the aforementioned methods is non-small cell lung cancer.

The invention further provides a pharmaceutical composition comprising emibetuzumab with one or more pharmaceutically acceptable carriers, diluents, or excipients, in combination with a pharmaceutical composition of an anti-MET inhibitor with one or more pharmaceutically acceptable carriers, diluents, or excipients. In a further embodiment, the anti-MET inhibitor is merestinib, or a pharmaceutically acceptable salt thereof.

In addition, the invention provides a kit comprising emibetuzumab in combination with an anti-MET inhibitor. The invention also provides a kit comprising emibetuzumab and merestinib, or a pharmaceutically acceptable salt thereof. The invention further provides a kit comprising a pharmaceutical composition comprising emibetuzumab with one or more pharmaceutically acceptable carriers, diluents, or excipients, and a pharmaceutical composition comprising an anti-MET inhibitor, with one or more pharmaceutically acceptable carriers, diluents, or excipients. The invention also provides a kit comprising a pharmaceutical composition comprising emibetuzumab, with one or more pharmaceutically acceptable carriers, diluents, or excipients, and a pharmaceutical composition comprising merestinib, or a pharmaceutically acceptable salt thereof, with one or more pharmaceutically acceptable carriers, diluents, or excipients.

The invention also provides a kit comprising an effective amount of emibetuzumab in combination with an effective amount of merestinib, or a pharmaceutically acceptable salt thereof, for simultaneous, separate or sequential use in the treatment of cancer with tumors bearing MET exon 14 skipping or MET exon 14 skipping phenotype. In a further embodiment of the invention, the cancer is lung, neuroblastoma, gastric, or colon cancer. More preferably, the cancer is gastric or lung cancer. Even more preferably, the cancer is carcinoma of the gastroesophageal junction. Even more preferably, the cancer is lung cancer. Most preferably, the cancer in the aforementioned methods is non-small cell lung cancer.

The invention further provides a combination comprising emibetuzumab and merestinib, or a pharmaceutically acceptable salt thereof, for simultaneous, separate or sequential use in the treatment of cancer with tumors bearing MET exon 14 skipping mutation(s) or MET exon 14 skipping phenotype. In a further embodiment of the invention, the cancer is lung, neuroblastoma, gastric, or colon cancer. More preferably, the cancer is gastric or lung cancer. Even more preferably, the cancer is carcinoma of the gastroesophageal junction. Even more preferably, the cancer is lung cancer. Most preferably, the cancer in the aforementioned methods is non-small cell lung cancer.

The invention further provides the use of a combination comprising emibetuzumab and meresitnib, or a pharmaceutically acceptable salt thereof, for use in therapy. The invention also provides a combination comprising emibetuzumab and merestinib, or a pharmaceutically acceptable salt thereof. A further embodiment of the invention is the use of the combination comprising emibetuzumab and merestinib, or a pharmaceutically acceptable salt thereof, for treating cancer with tumors bearing MET exon 14 skipping mutation(s) or MET exon 14 skipping phenotype. In a further embodiment of the invention, the cancer is lung, neuroblastoma, gastric, or colon cancer. More preferably, the cancer is gastric or lung cancer. Even more preferably, the cancer is carcinoma of the gastroesophageal junction. Even more preferably, the cancer is lung cancer. Most preferably, the cancer in the aforementioned methods is non-small cell lung cancer.

The invention further provides the use of a combination comprising emibetuzumab and an anti-MET inhibitor for the manufacture of a medicament for the treatment of cancer with tumors bearing MET exon 14 skipping or MET exon 14 skipping phenotype. In a further embodiment of the invention, the cancer is lung, neuroblastoma, gastric, or colon cancer. More preferably, the cancer is gastric or lung cancer. Even more preferably, the cancer is carcinoma of the gastroesophageal junction. Even more preferably, the cancer is lung cancer. Most preferably, the cancer in the aforementioned methods is non-small cell lung cancer.

Another aspect of the present invention is a method of treating cancer with tumors bearing MET exon 14 skipping mutation(s) or MET exon 14 skipping phenotype in a patient, comprising administering to a patient in need of such treatment wherein merestinib, or a pharmaceutically acceptable salt thereof, is administered at a dose between about 60 mg to about 160 mg daily on a 28-day cycle and emibetuzumab is administered at a dose between about 500 mg to about 2500 mg on about day 1 and about day 15 of the 28-day cycle of merestinib dosing. More preferably, merestinib, or a pharmaceutically acceptable salt thereof, is administered at a dose between about 80 mg to about 120 mg daily of a 28-day cycle and emibetuzumab is administered at a dose of about 750 mg on about day 1 and about day 15 of the 28-day cycle of the merestinib dosing. In yet a further embodiment, merestinib is administered orally and emibetuzumab is administered intravenously.

Each of the embodiments described herein envisions within its scope pharmaceutically acceptable salts of the compounds referenced or described herein. Accordingly, the phrase “or a pharmaceutically acceptable salt thereof” is implicit in the references to or descriptions of all compounds herein.

The phrase “MET exon 14 skipping mutation”, “exon 14 skipping mutation”, “MET exon 14 skipping”, “exon 14 skipping”, or grammatical versions thereof, as used herein, refer to somatic mutations in the gene for MET, which, upon translation of the mRNA transcripts expressed thereby, result in cellular expression of MET polypeptides wherein exon 14 is largely or entirely deleted.

The phrases “MET exon 14 skipping phenotype”, “exon 14 skipping phenotype”, or grammatical versions thereof, as used herein refer to any single somatic point mutation in the gene for MET which, upon translation of the mRNA transcripts expressed thereby, result in cellular expression of MET polypeptides mutated at Y1003, D1002 or R1004 and which have a diminished ability to bind the ubiquitin ligase CBL, resulting in a MET protein with increased stability and oncogenic potential.

The term “K_{D}”, as used herein, is intended to refer to the equilibrium dissociation constant of a particular antibody-antigen or antibody fragment-antigen interaction.

The phrase “specifically binds” as used herein in reference to the affinity of aantibody, or antigen-binding fragment thereof, for the MET-ECD is intended to mean, unless indicated otherwise, a K_{D} of less than about 1×10⁻⁸M, preferably, less than about 1×10⁻⁹M as determined by common methods known in the art, including by use of a surface plasmon resonance (SPR) biosensor at 25° C. (for MET-ECD).

As used herein, the term “C8-H241 antibody” or “C8-H241 Ab” refers to an antibody comprising: a LCVR whose amino acid sequence is that given in SEQ ID NO: 1, and a HCVR whose amino acid sequence is that given in SEQ ID NO: 3, wherein that C8-H241 Ab specifically binds to MET-ECD. In some embodiments, a C8-H241 Ab is a C8-H241-IgG4 Ab, an antibody comprising: a LC whose amino acid sequence is that given in SEQ ID NO: 5, and a HC whose amino acid sequence is that given in SEQ ID NO: 7 and that specifically binds to MET-ECD.

As used herein, the term “emibetuzumab” refers to a C8-H241-IgG4 antibody comprising: two light chains, each of whose amino acid sequence is that given in SEQ ID NO: 5, and two heavy chains, each of whose amino acid sequence is that given in SEQ ID NO: 7 and that specifically binds to MET-ECD (see, WHO Drug Information, Proposed International Nonproprietary Names (INN) List 111, Volume 28, No. 2, July 2014).

In certain embodiments, a C8-H241 Ab or a C8-H241-IgG4 Ab, including, but not limited to, emibetuzumab binds MET-ECD with a K_{D} value of between about 100 nM to about 1 pM, preferably, between about 10 nM to about 10 pM, more preferably, between about 10 nM to about 100 pM, more preferably, between about 2.5 nM to about 0.5 nM, and most preferably about 1 nM, as determined by a surface plasmon resonance (SPR) based assay (such as the BIAcore assay as described in PCT Application Publication No. WO 2005/012359) conducted at 25° C. Antibody affinities may also be determined by ELISA; and competition assays (e.g., a RIA), for example.

The terms “MET polypeptide”, “MET receptor”, “MET”, “HGF receptor” or “HGFR” are used interchangeably herein and, unless otherwise indicated, are intended to refer to the human receptor tyrosine kinase, as well as functionally active, mutated forms thereof, that bind human hepatocyte growth factor. Specific examples of MET include, e.g., a human polypeptide encoded by the nucleotide sequence provided in GenBank accession no. NM_000245, or the human protein encoded by the polypeptide sequence provided in GenBank accession no. NP_000236.

The extracellular domain of MET has the amino acid sequence shown in, for example, SEQ ID NO: 9. However, amino acids 1-24 of SEQ ID NO: 9 comprise the signal sequence. Therefore, unless stated otherwise, the term “MET-ECD” as used herein means the protein beginning and ending at amino acids 25 and 932, respectively, of SEQ ID NO: 9 (i.e., SEQ ID NO: 10). The SEMA domain consists of approximately 500 amino acid residues at the N-terminus of MET, and contains the α-chain (amino acid residues 25-307 of SEQ ID NO: 9 (i.e., SEQ ID NO: 11) and part of the β-chain (amino acid residues 308-519 of SEQ ID NO: 18 (i.e., SEQ ID NO: 12)).

Unless indicated otherwise, the term “antibody” refers to an immunoglobulin molecule comprising two heavy chains and two light chains interconnected by disulfide bonds. The amino terminal portion of each chain includes a variable region of about 100 to about 110 amino acids primarily responsible for antigen recognition via the complementarity determining regions (CDRs) contained therein. The carboxy-terminal portion of each chain defines a constant region primarily responsible for effector function.

As used herein, the term “antigen-binding fragment” refers to any antibody fragment that retains the ability to bind to its antigen. Such “antigen-binding fragments” can be selected from the group consisting of Fv, scFv, Fab, F(ab′)₂, Fab′, scFv-Fc fragments and diabodies. An antigen-binding fragment of an antibody will typically comprise at least one variable region. Preferably, an antigen-binding fragment comprises a heavy chain variable region (HCVR) and a light chain variable region (LCVR). More preferably, an antigen-binding fragment as used herein comprises a HCVR and a LCVR which confers antigen-binding specificity to MET-ECD (i.e., a “MET-ECD binding fragment”).

As used herein, the term “light chain variable region (LCVR)” refers to a portion of a LC of an antibody molecule that includes amino acid sequences of Complementarity Determining Regions (CDRs; i.e., LCDR1, LCDR2, and LCDR3), and Framework Regions (FRs).

As used herein, the term “heavy chain variable region (HCVR)” refers to a portion of a HC of an antibody molecule that includes amino acid sequences of Complementarity Determining Regions (CDRs; i.e., HCDR1, HCDR2, and HCDR3), and Framework Regions (FRs).

As used herein, the terms “complementarity determining region” and “CDR”, refer to the non-contiguous antigen combining sites found within the variable region of LC and HC polypeptides of an antibody or an antigen-binding fragment thereof. These particular regions have been described by others including Kabat, et al., Ann. NY Acad. Sci. 190:382-93 (1971); Kabat et al., J. Biol. Chem. 252:6609-6616 (1977); Kabat, et al., Sequences of Proteins of Immunological Interest, Fifth Edition, U.S. Department of Health and Human Services, NIH Publication No. 91-3242 (1991); Chothia, et al., J. Mol. Biol. 196:901-917 (1987); MacCallum, et al., J. Mol. Biol., 262:732-745 (1996); and North, et al., J. Mol. Biol., 406, 228-256 (2011), where the definitions include overlapping or subsets of amino acid residues when compared against each other.

The CDRs are interspersed with regions that are more conserved, termed framework regions (“FR”). Each LCVR and HCVR is composed of three CDRs and four FRs, arranged from amino-terminus to carboxy-terminus in the following order: FR1, CDR1, FR2, CDR2, FR3, CDR3, FR4. The three CDRs of the light chain are referred to as “LCDR1, LCDR2, and LCDR3” and the three CDRs of the HC are referred to as “HCDR1, HCDR2, and HCDR3.” The CDRs contain most of the residues which form specific interactions with the antigen. The numbering and positioning of CDR amino acid residues within the LCVR and HCVR regions is in accordance with known conventions (e.g., Kabat (1991), Chothia (1987), and/or North (2011)). In different embodiments of the invention, the FRs of the antibody may be identical to the human germline sequences, or may be naturally or artificially modified.

In certain embodiments, C8-H241 antibodies, including C8-H241-IgG4 antibodies or emibetuzumab, for the methods and/or uses of the present invention may be altered to increase or decrease the extent to which the antibody is glycosylated. Addition or deletion of glycosylation sites to an antibody may be conveniently accomplished by altering the amino acid sequence such that one or more glycosylation sites is created or removed.

Where a C8-H241 antibody, including, but not limited to, emibetuzumab, comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region (see, e.g., Wright et al. TIBTECH 15:26-32 (1997)). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In some embodiments, the C8-H241 antibody, including, but not limited to, emibetuzumab may have a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycosyl structures attached to Asn297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function (see, US Patent Publication Nos. US 2003/0157108 and US 2004/0093621, for example). Examples of publications related to “defucosylated” or “fucose-deficient” antibody variants include: US 2003/0157108; WO 2000/61739; WO 2001/29246; US 2003/0115614; US 2002/0164328; and Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1; and WO 2004/056312 A1), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO 2003/085107).

Unless indicated otherwise, when referring to an amino acid residue in an antibody by a number, the EU numbering system is used herein as it is conventionally used in the art (see, Kabat, et al., Sequences of Proteins of Immunological Interest, Fifth Edition, U.S. Department of Health and Human Services, NIH Publication No. 91-3242 (1991), for example).

As used herein, the term “kit” refers to a package comprising at least two separate containers, wherein a first container contains a C8-H241 antibody, preferably, emibetuzumab, and a second container contains an anti-MET inhibitor. As used herein, the term “kit” also refers to a package comprising at least two separate containers, wherein a first container contains emibetuzumab, and a second container contains merestinib, or a pharmaceutically acceptable salt thereof. A “kit” may also include instructions to administer all or a portion of the contents of these first and second containers to a cancer patient, preferably, a gastric cancer patient, a patient having carcinoma of the GEJ, a lung cancer patient, or a NSCLC patient and preferably a cancer patient with tumors with MET exon 14 skipping mutation(s) or MET exon 14 skipping phenotype.

As used herein, the terms “treating,” “to treat,” or “treatment” refers to restraining, slowing, stopping, reducing, or reversing the progression or severity of an existing symptom, disorder, condition, or disease.

As used herein, the term “patient” refers to a mammal, preferably a human.

As used herein, the terms “cancer” and “cancerous” refer to or describe the physiological condition in patients that is typically characterized by unregulated cell proliferation. Included in this definition are benign and malignant cancers. By “early stage cancer” or “early stage tumor” is meant a cancer that is not advanced or metastatic or is classified as a Stage 0, I, or II cancer. Examples of cancer include, but are not limited to, gastric cancer, preferably, carcinoma of the gastroesophageal junction, and lung cancer, preferably NSCLC.

In some embodiments of the present invention, the cancer patients are selected for treatment with a combination therapy disclosed herein on the basis of having a tumor in which MET is expressed or overexpressed. Preferably, the MET expression status of a cancer patient's tumor is determined by using an immunohistochemistry (IHC) assay, PCR assay, gene sequencing assay and/or fluorescence in-situ hybridization (FISH) assay suitable for the detection of MET or MET gene amplification. More preferably, an IHC method for determining whether a cancer patient's tumor expresses or overexpresses MET is performed essentially as described in Example 7 of PCT International Publication WO 2013/169532 using an anti-MET antibody, or an antigen-binding fragment thereof, disclosed therein that specifically binds to MET-ECD, wherein said anti-MET antibody comprises a LC and a HC, wherein the amino acid sequence of the LC and HC is that given in SEQ ID NO: 13 and SEQ ID NO: 14, respectively. In some embodiments, a patient is selected for treatment with the combination therapies of the present invention after a sample of a cancer patient's tumor has been determined expressed or overexpress MET by use of an IHC assay wherein the assay comprises the step of contacting the sample with a MET antibody, or antigen-binding fragment thereof, wherein the antibody, or fragment thereof, comprises a LC and a HC, wherein the amino acid sequence of the LC and HC is that given in SEQ ID NO: 13 and SEQ ID NO: 14, respectively. In various embodiments of the methods of the present invention, the aforementioned IHC assay of the patient's tumor is performed using a formalin-fixed and paraffin-embedded sample of the patient's tumor.

In some embodiments of the present invention, the cancer patients are selected for treatment with a combination therapy disclosed herein on the basis of having a tumor with MET exon 14 skipping mutations or MET exon 14 phenotype. Preferably, the MET exon 14 skipping mutation status of a cancer patient's tumor is determined by next generation gene sequencing methodologies. More preferably, the MET exon 14 skipping mutations or MET exon 14 phenotype of a cancer patient's tumor is determined by using Hybridization-captured Next Generation Sequencing (see., e.g., Schrock, A. B., et al., J Thoracic Oncology 2016, 9(11): 1493-1502). More preferably, the MET exon 14 phenotype of a cancer patient's tumor is determined by using the nCounter Analysis System (NANOSTRING® Technologies), a fluorescence-based platform for multiplexed digital mRNA profiling without amplification or generation of cDNA (see., e.g., Geiss, G. K., et al., Nature Biotechnology 2008, 26: 317-325).

A main advantage of the combination treatments of the invention is the ability of producing marked anti-cancer effects in a patient without causing significant toxicities or adverse events, so that the patient benefits from the combination treatment method overall. The efficacy of the combination treatment of the invention can be measured by various endpoints commonly used in evaluating cancer treatments, including but not limited to, tumor regression, tumor weight or size shrinkage, time to progression, overall survival, progression free survival, overall response rate, duration of response, and quality of life. The therapeutic agents used in the invention may cause inhibition of metastatic spread without shrinkage of the primary tumor, may induce shrinkage of the primary tumor, or may simply exert a tumoristatic effect. Because the invention relates to the use of a combination of unique anti-tumor agents, novel approaches to determining efficacy of any particular combination therapy of the present invention can be optionally employed, including, for example, measurement of plasma or urinary markers of angiogenesis and measurement of response through radiological imaging.

As used herein, the term “effective amount” refers to the amount or dose of a C8-H241 antibody, preferably, emibetuzumab, and/or to the amount or dose of an anti-MET inhibitor, preferably, merestinib, which, upon single or multiple dose administration to the patient, optionally in combination with one or more other active agents, as the case may be, provides an effective response in the patient under diagnosis or treatment. As used herein, the term “effective amount” also refers to the amount or dose of a C8-H241 antibody, preferably, emibetuzumab, and to the amount or dose of merestinib, or a pharmaceutically acceptable salt thereof, which, upon single or multiple dose administration to the patient, optionally, in combination with one or more other active agents, provides an effective response in the patient under diagnosis or treatment. It is understood that a combination therapy of the present invention is carried out by administering a C8-H241 antibody, preferably, emibetuzumab, together with an anti-MET inhibitor in any manner which provides effective levels of the C8-H241 antibody, preferably, emibetuzumab, and the anti-MET inhibitor, prefereably, merestinib, in the body. It is also understood that a combination therapy of the present invention is carried out by administering emibetuzumab together with merestinib, or a pharmaceutically acceptable salt thereof, in any manner which provides effective levels of emibetuzumab and merestinib in the body.

As used herein, the terms “effective response” of a patient or a patient's “responsiveness” to treatment with a combination of agents, or “therapeutic effect” refers to the clinical or therapeutic benefit(s) imparted to a patient upon administration of i) a combination of a C8-H241 antibody, preferably, emibetuzumab, and an anti-MET inhibitor, or ii) emibetuzumab and merestinib, or a pharmaceutically acceptable salt thereof. Such benefit(s) include any one or more of: extending survival (including overall survival and progression free survival); resulting in an objective response (including a complete response or a partial response); tumor regression, tumor weight or size shrinkage, longer time to disease progression, increased duration of survival, longer progression free survival, improved overall response rate, increased duration of response, and improved quality of life and/or improving signs or symptoms of cancer, etc.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

Emibetuzumab is generally effective over a wide dosage range in the combination of the present invention. For example, dosages normally are given on days one and fifteen of a 28-day treatment cycle and each dose falls within the range of about 500 mg to about 2500 mg, preferably about 750 mg to about 2000 mg, and most preferably about 750 mg. In addition, an anti-MET inhibitor, preferably merestinib, or a pharmaceutically acceptable salt thereof, is generally effective over a wide dosage range in the combination of the present invention. For example, dosages per 28-day cycle normally fall within the range of daily dosing of about 60 to 160 mg, preferably about 80 to about 120 mg, and most preferably 120 mg. In some instances dosage levels below the lower limit of the aforesaid ranges for emibetuzumab and merestinib, or a pharmaceutically acceptable salt thereof, may be more than adequate, while in other cases smaller or still larger doses may be employed with acceptable side effects, and therefore the above dosage range is not intended to limit the scope of the invention in any way. When given in combination with an anti-MET inhibitor, for example, over a 28-day cycle, emibetuzumab, is administered on days one and fifteen within the range of about 500 mg to about 2500 mg and an anti-MET inhibitor, preferably, merestinib, or a pharmaceutically acceptable salt thereof, is administered daily within the range of about 60 to 120 mg. When given in combination, for example, over a 28-day cycle, emibetuzumab is administered on day one and day fifteen within the range of about 750 mg to about 2000 mg and an anti-MET inhibitor, preferably, merestinib, or a pharmaceutically acceptable salt thereof, is administered on day one and day fifteen within the range of about 80-120 mg. Optionally, a 21-day cycle may be employed with dose given daily the dosage of merestinib, or a pharmaceutically acceptable salt thereof, within the range of about 60-120 mg, more preferably, 80-120 mg and the dosage of emibetuzumab in the range of about 750 mg to about 200 mg, more preferably, 1000 mg given on day one and day 21.

Emibetuzumab, and an anti-MET inhibitor, preferably, merestinib, or a pharmaceutically acceptable salt thereof, are preferably formulated as pharmaceutical compositions administered by any route which makes the compound bioavailable. The route of administration may be varied in any way, limited by the physical properties of the drugs and the convenience of the patient and the caregiver. Preferably, an anti-MET inhibitor, more preferably, merestinib, or a pharmaceutically acceptable salt thereof, compositions are for oral administration, although parenteral administration, including intravenous or subcutaneous administration, may be used. (See, e.g., Remington: The Science and Practice of Pharmacy (D. B. Troy, Editor, 21st Edition, Lippincott, Williams & Wilkins, 2006.) Pharmaceutical compositions of emibetuzumab are for parenteral administration, such as intravenous or subcutaneous administration. More preferably, emibetuzumab compositions comprise about 10 to about 20 mg/ml of emibetuzumab, about 10 to about 20 mM histidine buffer, pH 5.5-6.0, about 75 mM to about 150 mM sodium chloride, about 0.01% to about 0.06% polysorbate 80, and, optionally, about 100 mM to about 150 mM glycine. Preferably, emibetuzumab compositions are formulated for intravenous administration and comprise about 20 mg/ml of emibetuzumab, about 10 mM histidine buffer, pH 5.5, about 150 mM sodium chloride, and about 0.06% polysorbate 80. Such pharmaceutical compositions and processes for preparing same are well known in the art. (See, e.g., Remington: The Science and Practice of Pharmacy (D. B. Troy, Editor, 21st Edition, Lippincott, Williams & Wilkins, 2006.)

As used herein, the term “in combination with” refers to the administration of an nti-MET inhibitor, preferably merestinib, or a pharmaceutically acceptable salt thereof, and emibetuzumab either separately, simultaneously, or sequentially in any order, such as for example, at repeated intervals as during a standard course of treatment for a single cycle or more than one cycle, such that one agent can be administered prior to, at the same time, or subsequent to the administration of the other agent, or any combination thereof. It can also mean that one drug is given alone first until patients progress before the second drug is being added on.

A main advantage of the combination treatments of the invention is the ability of producing marked anti-cancer effects in a patient without causing significant toxicities or adverse events, so that the patient benefits from the combination treatment method overall. The efficacy of the combination treatment of the invention can be measured by various endpoints commonly used in evaluating cancer treatments, including but not limited to, tumor regression, tumor weight or size shrinkage, time to progression, overall survival, progression free survival, overall response rate, duration of response, and quality of life. The therapeutic agents used in the invention may cause inhibition of metastatic spread without shrinkage of the primary tumor, may induce shrinkage of the primary tumor, or may simply exert a tumoristatic effect. Because the invention relates to the use of a combination of unique anti-tumor agents, novel approaches to determining efficacy of any particular combination therapy of the present invention can be optionally employed, including, for example, measurement of plasma or urinary markers of angiogenesis and/or cell cycle activity, tissue-based biomarkers for angiogenesis and/or cell cycle activity and/or circulating levels of MET exon 14 skipping polypeptides and/or MET mutations leading to MET exon 14 skipping or exon 14 skipping phenotye, and measurement of response through radiological imaging.

The following examples illustrate the unexpected benefit of the present combinations.

### EXAMPLE 1

### Evaluation of the Combination of Merestinib (LY2801653) and Emibetuzumab (LY2875358) in a Xenograft Tumor Model Bearing MET Exon 14 Skipping Mutation(s)

To determine the efficacy of merestinib, in combination with emibetuzumab, in an Hs746t-derived xenograft mouse model of human gastric carcinoma and to compare the combination effect to that of either monotherapy, studies conducted essentially as described below may be performed. Hs746t is a gastric cancer cell line known to have MET exon 14 skipping mutation(s) and MET amplification (Asaoka et al., Biochem Biophys Res Commun 394:1042-1046).

### Study Designs and Methods:

Female athymic nude mice (Envigo) are used for this study. Food and water are available ad libitum. Animals are acclimated for 1 week prior to any experimental manipulation. The study is performed in accordance with AAALAC accredited institutional guidelines.

Merestinib is formulated as a solution in 10% PEG 400/90% (20% Captisol in water). Emibetuzumab is diluted in phosphate buffered saline (PBS). Solutions are freshly prepared every 7 days.

Obtain Hs746t cells from ATCC (American Type Culture Collection) and maintain in RPMI 1640 with 10% fetal bovine serum at 37° C. in 5% CO₂. Expand cells in culture, harvest, and wash in Hank's Balanced Salt Solution (HBSS, GIBC®). Implant sub-confluent MKN45 cells into 60 female nu/nu mice at a concentration of 1×10⁷ cells in 0.2 ml HBSS subcutaneously into the hind flank of the animal. Randomize the animals when tumors reach an average volume of 350 mm³ by tumor volume into four treatment groups (n=7): PBS vehicle control; merestinib (at 6 mg/kg or 12 mg/kg), qd orally; emibetuzumab, at 10 mg/kg, IP, qw; merestinib (at 6 mg/kg or 12 mg/kg) qd orally and emibetuzumab at 10 mg/kg, IP, qw.

Administer merestinib by oral gavage at either 6 mg/kg or 12 mg/kg in a dose volume of 0.2 mL in 10% polyethylene glycol 400/90% of a mixture of 20% Captisol in water, and antibody treatments by intraperitoneal injection (IP) starting on day 12 after tumor cell implantation when average tumor volume of 350 mm³ and ending on Day 33. Inject animals IP in a dosing volume of 10 mg/kg. After 14 days of dosing (Study Day 27), the animals in the control group are sacrificed because of tumor volumes exceeding >2000 mm³. After the 28-day dosing period, animals in the single agent arms are sacrificed. Tumors in the animals in the combination treatment groups are observed for an additional 2 weeks.

In both studies, animals are sacrificed using CO₂ and cervical dislocation when tumors grow larger than 2000 mm³, or at the end of study.

Dose animals in treatment group 4, with merestinib first, followed by emibetuzumab, 30-45 minutes later. Record tumor growth and body weight changes at least twice a week. Body weight measurement during the course of the study, is a general indicator of tolerability.

Measure tumor growth with calipers and record body weights twice weekly. Calculate tumor volumes by the formula Volume (mm³)=L×W² (π/6) where L represents the larger diameter and W the smaller diameter. Calculate T/C % using the formula T/C %=100×ΔT/ΔC. Where ΔT=mean tumor volume of the drug-treated group on the final day of the study−mean tumor volume of the drug-treated group on the initial day of the dosing and ΔC=mean tumor volume of the control group on the final day of the study−mean tumor volume of the control group on the initial day of the dosing. Calculate changes in body weight by the formula (Weight on observation day−Weight on day 12)/Weight on Day 12×100. Calculate test for significant differences between treatment groups by RMANOVA using the JMP (v.9.0.3) statistical package (SAS Institute Inc., Cary, N.C., USA).

### Results:

Merestinib inhibits Hs746t cell proliferation with IC₅₀=34 nM and totally eliminates pMET at 65-100 nM. Emibetuzumab slightly inhibits Hs746t cell proliferation (IC₅₀>100 nM), reduces 10-20% cell surface MET, and shows no effect on pMET expression (at 130-650 nM).

In the Hs746t xenograft model, merestinib (12 mg/kg) treatment results in 91.8% tumor regression after 21 day dosing, while 6 mg/kg merestinib provides transient tumor regression followed by re-growth while on treatment with T/C=18.3% after 21 day dosing. No tumor regrowth is observed in 6/7 mice in the 12 mg/kg merestinib cohort during the 5 weeks post-treatment. Emibetuzumab treatment provides transient tumor regression (37.7%) after 3 doses, but tumors are observed to regrow while on treatment. Combination of 6 mg/kg merestinib and 10 mg/kg emibetuzumab results in 85% tumor regression for the duration of the 28 day dosing period and the treatment is well tolerated. Regrowth of tumors in animals is observed upon termination of this combination treatment.

All animals are shown to have survived the dosing and observation period. No loss in body weight is observed in the combination treatment group. All mice in the vehicle control group display disease progression.

To assess the effect of the combination of merestinib and emibetuzumab, a repeated measures ANOVA model is fit on log transformed tumor volume using the mixed procedure in SAS software (version 9.3, Cary, N.C.) followed by a 2×2 interaction test to test for statistical significance of the combination of the two single agents. The observed percent reduction of the combination on day 28 is 83.1%. The expected percent reduction of the combination on day 28 is 81.5%. The p-value from the 2×2 interaction test on day 28 is not significant at p=0.654. All pairwise comparisons between the combination group and each single agent group on day 33 are also statistically significant. By inspection of the means, these results indicate that the combination group is statistically smaller than each single agent group. Full statistical analysis is not feasible as the animals in the vehicle control groups were removed after 14 days of dosing as the animals are sacrificed due to tumor burden.

The data of Example 1 indicates that the combination of merestinib and emibetuzumab results in tumor regression.

### EXAMPLE 2

### Evaluation of Sequential and Concurrent Administration of Merestinib (LY2801653) and Emibetuzumab (LY2875358) in a Xenograft Tumor Model Bearing MET Exon 14 Skipping Mutation(s)

To determine the efficacy of low-dose merestinib, in sequential and concurrent combination with emibetuzumab, in an Hs746t-derived xenograft mouse model of human gastric carcinoma, and to compare the combination effect to that of higher-dose emibetuzumab monotherapy, studies conducted essentially as described below may be performed.

### Study Designs and Methods:

Female athymic nude mice (Envigo) are used for this study. Food and water are available ad libitum. Animals are acclimated for 1 week prior to any experimental manipulation. The study is performed in accordance with AAALAC accredited institutional guidelines.

Merestinib is formulated as a solution in 10% PEG 400/90% (20% Captisol in water). Emibetuzumab and IgG control are diluted in PBS. Solutions are freshly prepared every 7 days.

Obtain, maintain, expand, harvest, and wash Hs746t cell as in Example 1. Implant cells into 36 female nu/nu mice at a concentration of 2×10⁶ cells in 0.2 ml HBSS subcutaneously into the hind flank of the animal. Randomize the animals (N=36) when tumors reach an average volume of 350 mm³ by tumor volume into five treatment groups (n=6): combined vehicle control of 10% PEG 400/90% (20% Captisol in water) administered by oral gavage qd, and IgG control at 10 mg/kg administered via IP injection qw; emibetuzumab (10 mg/kg and 20 mg/kg) administered via IP injections qw for 4 cycles; emibetuzumab (10 mg/kg) administered via IP qw for 7 cycles with concurrent administration of merestinib (6 mg/kg) administered orally qd for 50 days; and merestininb (6 mg/kg), administered orally qd for 17 days, as single agent, with sequential combination administration of emibetuzumab (10 mg/kg) administered via IP qw for 6 cycles starting on day 37 (whereupon tumor regression and regrowth to 500 mm³ after single agent merestinib (day 37) is observed), for a total of 58 days of merestinib dosing. After the 28-day dosing period, animals in the single agent arms are sacrificed. Tumors in the animals in the combination treatment groups are monitored for continued growth or regrowth of tumors.

In these studies, animals are sacrificed using CO₂ and cervical dislocation when tumors grow larger than 2000 mm³, or at the end of study.

Measure tumor volumes and body weight at least twice weeky. Body weight measurement during the course of the study, is a general indicator of tolerability.

Measure tumor growth with calipers and record body weights twice weekly. Calculate tumor volumes by the formula Volume (mm³)=L×W² (π/6) where L represents the larger diameter and W the smaller diameter. Calculate T/C % using the formula T/C %=100×ΔT/ΔC. Where ΔT=mean tumor volume of the drug-treated group on the final day of the study−mean tumor volume of the drug-treated group on the initial day of the dosing and ΔC=mean tumor volume of the control group on the final day of the study−mean tumor volume of the control group on the initial day of the dosing. Calculate changes in body weight by the formula (Weight on observation day−Weight on day 12)/Weight on Day 12×100. Calculate test for significant differences between treatment groups by RM ANOVA using the JMP (v.9.0.3) statistical package (SAS Institute Inc., Cary, N.C., USA).

Transform tumor volume to the log scale to equalize variance across time and treatment groups. Analyze the log volume data with a two-way repeated measures analysis of variance by time and treatment using the MIXED procedures in SAS software (Version 9.3). The correlation model for the repeated measures is spatial power. Compare treated groups to the control group at each time point. The MIXED procedure is also used separately for each treatment group to calculate adjusted means and standard errors at each time point. Both analyses account for the autocorrelation within each animal and the loss of data that occurs when animals are removed or lost before the end of the study. The adjusted means and standard errors are plotted for each treatment group versus time. Full statistical analysis is not feasible as the animals in the vehicle control groups were removed after 17 days of dosing (study day 37), as half the animals from the vehicle group are removed and sacrificed due to tumor burden (tumor volume>2000 mm³).

### Results:

In the Hs746t xenograft model, merestinib (6 mg/kg qd for 58 days) results in transient tumor regression, with maximal regression (30.2%) occurring on Day 27 (7 days of dosing), followed by re-growth while on treatment; by Day 37, tumor volumes approach about 500 mm³. Sequential addition of emibetuzumab (10 mg/kg qw for 6 cycles) to merestinib treatment on Day 37 results in tumor regression in 5/6 animals, with complete regression and no tumor regrowth over a 26 day observation period after dosing completion in 2/6 animals. Transient tumor regression with slow regrowth during post-treatment observation is evident in an additional 2/6 animals. In an additional animal, tumor regression occurs to about 50 mm³ with slow regrowth during the post-treatment observation. In the one non-responding animal, tumor size on day 37 is notably the largest (approximately 1200 mm³) in the entire cohort, when emibetuzumab treatment is initiated.

Concurrent treatment with merestinib (6 mg/kg qd for 50 days) and emibetuzumab (10 mg/kg qw for 7 cycles) results in initial tumor regression (87.5% on day 37), but regression proved transient, with slow tumor regrowth over the course of treatment. Tumor size approaches pre-treatment volumes by Day 70, and additional monitoring for a week post-treatment results in tumor size doubling in volume in 4/6 animals.

Single-agent emibetuzumab treatment (10 mg/kg and 20 mg/kg qw 28 days) results in transient tumor regression at both doses. In the 10 mg/kg cohort, maximum regression (16.0%) is observed on Day 27, with maximum regression (58.9%) in the 20 mg/kg cohort occurring on Day 37. Tumor volumes from both cohorts were not statistically significant (p=0.129).

In these studies, body weight measurements provide an indication of the tolerability of the various treatments. There were no significant decreases in body weight observed in any group.

The data of Example 2 indicates that the concurrent combination of low-dose merestinib and emibetuzumab results in transient tumor regression, with sequential combination of emibetuzumab to low-dose merestinib resulting in longer duration of treatment response.

SEQUENCE LISTING
<SEQ ID NO: 1; PRT1; Artificial Sequence> Emibetuzumab LCVR
DIQMTQSPSSLSASVGDRVTITCSVSSSVSSIYLHWYQQKPGKAPKLLIYSTSNLASGVPSRFSG
SGSGTDFTLTISSLQPEDFATYYCQVYSGYPLTFGGGTKVEIK
<SEQ ID NO: 2; DNA; Artificial Sequence> Emibetuzumab DNA LCVR
GACATCCAGATGACCCAGTCTCCATCCTCCCTGTCTGCATCTGTAGGAGACAGAGTCACCATCAC
TTGCAGTGTCAGCTCAAGTGTATCCTCCATTTACTTGCACTGGTATCAGCAGAAACCAGGGAAAG
CCCCTAAGCTCCTGATCTATAGCACATCCAACTTGGCTTCTGGAGTCCCATCAAGGTTCAGTGGC
AGTGGATCTGGGACAGATTTCACTCTCACCATCAGCAGTCTGCAACCTGAAGATTTTGCAACTTA
CTACTGTCAAGTCTACAGTGGTTACCCGCTCACGTTCGGCGGAGGGACCAAGGTGGAGATCAAA
<SEQ ID NO: 3; PRT1; Artificial Sequence> Emibetuzumab HCVR
QVQLVQSGAEVKKPGASVKVSCKASGYTFTDYYMHWVRQAPGQGLEWMGRVNPNRRGTTYNQKFE
GRVTMTTDTSTSTAYMELRSLRSDDTAVYYCARANWLDYWGQGTTVTVSS
<SEQ ID NO: 4; DNA; Artificial Sequence> Emibetuzumab DNA HCVR
CAGGTTCAGCTGGTGCAGTCTGGTGCTGAGGTGAAGAAGCCTGGTGCCTCAGTGAAGGTCTCCTG
CAAGGCTTCTGGTTACACCTTTACCGACTACTACATGCACTGGGTGCGTCAGGCCCCTGGTCAAG
GTCTTGAGTGGATGGGTCGTGTTAATCCTAACCGGAGGGGTACTACCTACAACCAGAAATTCGAG
GGCCGTGTCACCATGACCACAGACACATCCACGAGCACAGCCTACATGGAGCTGCGTAGCCTGCG
TTCTGACGACACGGCCGTGTATTACTGTGCGCGTGCGAACTGGCTTGACTACTGGGGCCAGGGCA
CCACCGTCACCGTCTCC
<SEQ ID NO: 5; PRT1; Artificial Sequence> Emibetuzumab LC
DIQMTQSPSSLSASVGDRVTITCSVSSSVSSIYLHWYQQKPGKAPKLLIYSTSNLASGVPSRFSG
SGSGTDFTLTISSLQPEDFATYYCQVYSGYPLTFGGGTKVEIKRTVAAPSVFIFPPSDEQLKSGT
ASVVCLLNNFYPREAKVQWKVDNALQSGNSQESVTEQDSKDSTYSLSSTLTLSKADYEKHKVYAC
EVTHQGLSSPVTKSFNRGEC
<SEQ ID NO: 6; DNA; Artificial Sequence> Emibetuzumab DNA LC
GACATCCAGATGACCCAGTCTCCATCCTCCCTGTCTGCATCTGTAGGAGACAGAGTCACCATCAC
TTGCAGTGTCAGCTCAAGTGTATCCTCCATTTACTTGCACTGGTATCAGCAGAAACCAGGGAAAG
CCCCTAAGCTCCTGATCTATAGCACATCCAACTTGGCTTCTGGAGTCCCATCAAGGTTCAGTGGC
AGTGGATCTGGGACAGATTTCACTCTCACCATCAGCAGTCTGCAACCTGAAGATTTTGCAACTTA
CTACTGTCAAGTCTACAGTGGTTACCCGCTCACGTTCGGCGGAGGGACCAAGGTGGAGATCAAAC
GAACTGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGGA
TAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGGAGAGTGC
<SEQ ID NO: 7; PRT1; Artificial Sequence> Emibetuzumab HC
QVQLVQSGAEVKKPGASVKVSCKASGYTFTDYYMHWVRQAPGQGLEWMGR
VNPNRRGTTYNQKFEGRVTMTTDTSTSTAYMELRSLRSDDTAVYYCARAN
WLDYWGQGTTVTVSSASTKGPSVFPLAPCSRSTSESTAALGCLVKDYFPE
PVTVSWNSGALTSGVHTFPAVLQSSGLYSLSSVVTVPSSSLGTKTYTCNV
DHKPSNTKVDKRVESKYGPPCPPCPAPEAAGGPSVFLFPPKPKDTLMISR
TPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFF
LYSRLTVDKSRWQEGNVFSCSVMHEALHNHYTQKSLSLSLG
<SEQ ID NO: 8; DNA; Artificial Sequence> Emibetuzumab HC
CAGGTTCAGCTGGTGCAGTCTGGTGCTGAGGTGAAGAAGCCTGGTGCCTCAGTGAA
GGTCTCCTGCAAGGCTTCTGGTTACACATTCACTGACTACTACATGCACTGGGTGCG
TCAGGCCCCTGGTCAAGGTCTTGAGTGGATGGGTCGTGTTAATCCTAACCGGAGGG
GTACTACCTACAACCAGAAATTCGAGGGCCGTGTCACCATGACCACAGACACATC
CACGAGCACAGCCTACATGGAGCTGCGTAGCCTGCGTTCTGACGACACGGCCGTGT
ATTACTGTGCGCGTGCGAACTGGCTTGACTACTGGGGCCAGGGCACCACCGTCACC
GTCTCCTCCGCCTCCACCAAGGGCCCATCGGTCTTCCCGCTAGCGCCCTGCTCCAGG
AGCACCTCCGAGAGCACAGCCGCCCTGGGCTGCCTGGTCAAGGACTACTTCCCCG
AACCGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCGTGC
CCTCCAGCAGCTTGGGCACGAAGACCTACACCTGCAACGTAGATCACAAGCCCAG
CAACACCAAGGTGGACAAGAGAGTTGAGTCCAAATATGGTCCCCCATGCCCACCC
TGCCCAGCACCTGAGGCCGCCGGGGGACCATCAGTCTTCCTGTTCCCCCCAAAACC
CAAGGACACTCTCATGATCTCCCGGACCCCTGAGGTCACGTGCGTGGTGGTGGACG
TGAGCCAGGAAGACCCCGAGGTCCAGTTCAACTGGTACGTGGATGGCGTGGAGGT
GCATAATGCCAAGACAAAGCCGCGGGAGGAGCAGTTCAACAGCACGTACCGTGT
GGTCAGCGTCCTCACCGTCCTGCACCAGGACTGGCTGAACGGCAAGGAGTACAAG
TGCAAGGTCTCCAACAAAGGCCTCCCGTCCTCCATCGAGAAAACCATCTCCAAAGC
CAAAGGGCAGCCCCGAGAGCCACAGGTGTACACCCTGCCCCCATCCCAGGAGGA
GATGACCAAGAACCAGGTCAGCCTGACCTGCCTGGTCAAAGGCTTCTACCCCAGC
GACATCGCCGTGGAGTGGGAAAGCAATGGGCAGCCGGAGAACAACTACAAGACC
ACGCCTCCCGTGCTGGACTCCGACGGCTCCTTCTTCCTCTACAGCAGGCTAACCGTG
GACAAGAGCAGGTGGCAGGAGGGGAATGTCTTCTCATGCTCCGTGATGCATGAGG
CTCTGCACAACCACTACACACAGAAGAGCCTCTCCCTGTCTCTGGGT
<SEQ ID NO: 9; PRT; Homo sapiens> Extracellular domain of MET
MKAPAVLAPGILVLLFTLVQRSNGECKEALAKSEMNVNMKYQLPNFTAETPIQNVILHEH
HIFLGATNYIYVLNEEDLQKVAEYKTGPVLEHPDCFPCQDCSSKANLSGGVWKDNINMAL
VVDTYYDDQLISCGSVNRGTCQRHVFPHNHTADIQSEVHCIFSPQIEEPSQCPDCVVSAL
GAKVLSSVKDRFINFFVGNTINSSYFPDHPLHSISVRRLKETKDGFMFLTDQSYIDVLPE
FRDSYPIKYVHAFESNNFIYFLTVQRETLDAQTFHTRIIRFCSINSGLHSYMEMPLECIL
TEKRKKRSTKKEVFNILQAAYVSKPGAQLARQIGASLNDDILFGVFAQSKPDSAEPMDRS
AMCAFPIKYVNDFFNKIVNKNNVRCLQHFYGPNHEHCFNRTLLRNSSGCEARRDEYRTEF
TTALQRVDLFMGQFSEVLLTSISTFIKGDLTIANLGTSEGRFMQVVVSRSGPSTPHVNFL
LDSHPVSPEVIVEHTLNQNGYTLVITGKKITKIPLNGLGCRHFQSCSQCLSAPPFVQCGW
CHDKCVRSEECLSGTWTQQICLPAIYKVFPNSAPLEGGTRLTICGWDFGFRRNNKFDLKK
TRVLLGNESCILTLSESTMNTLKCTVGPAMNKHFNMSIIISNGHGTTQYSTFSYVDPVIT
SISPKYGPMAGGILLTLIGNYLNSGNSRHISIGGKTCTLKSVSNSILECYTPAQTISTEF
AVKLKIDLANRETSIFSYREDPIVYEIHPTKSFISGGSTITGVGKNLNSVSVPRMVINVH
EAGRNFTVACQHRSNSEIICCTTPSLQQLNLQLPLKTKAFFMLDGILSKYFDLIYVHNPV
FKPFEKPVMISMGNENVLEIKGNDIDPEAVKGEVLKVGNKSCENIHLHSEAVLCTVPNDL
LKLNSELNIEWKQAISSTVLGKVIVQPDQNFT
<SEQ ID NO: 10; PRT; Homo sapiens> Mature MET-ECD
ECKEALAKSEMNVNMKYQLPNFTAETPIQNVILHEH
HIFLGATNYIYVLNEEDLQKVAEYKTGPVLEHPDCFPCQDCSSKANLSGGVWKDNINMAL
VVDTYYDDQLISCGSVNRGTCQRHVFPHNHTADIQSEVHCIFSPQIEEPSQCPDCVVSAL
GAKVLSSVKDRFINFFVGNTINSSYFPDHPLHSISVRRLKETKDGFMFLTDQSYIDVLPE
FRDSYPIKYVHAFESNNFIYFLTVQRETLDAQTFHTRIIRFCSINSGLHSYMEMPLECIL
TEKRKKRSTKKEVFNILQAAYVSKPGAQLARQIGASLNDDILFGVFAQSKPDSAEPMDRS
AMCAFPIKYVNDFFNKIVNKNNVRCLQHFYGPNHEHCFNRTLLRNSSGCEARRDEYRTEF
TTALQRVDLFMGQFSEVLLTSISTFIKGDLTIANLGTSEGRFMQVVVSRSGPSTPHVNFL
LDSHPVSPEVIVEHTLNQNGYTLVITGKKITKIPLNGLGCRHFQSCSQCLSAPPFVQCGW
CHDKCVRSEECLSGTWTQQICLPAIYKVFPNSAPLEGGTRLTICGWDFGFRRNNKFDLKK
TRVLLGNESCILTLSESTMNTLKCTVGPAMNKHFNMSIIISNGHGTTQYSTFSYVDPVIT
SISPKYGPMAGGILLTLIGNYLNSGNSRHISIGGKTCTLKSVSNSILECYTPAQTISTEF
AVKLKIDLANRETSIFSYREDPIVYEIHPTKSFISGGSTITGVGKNLNSVSVPRMVINVH
EAGRNFTVACQHRSNSEIICCTTPSLQQLNLQLPLKTKAFFMLDGILSKYFDLIYVHNPV
FKPFEKPVMISMGNENVLEIKGNDIDPEAVKGEVLKVGNKSCENIHLHSEAVLCTVPNDL
LKLNSELNIEWKQAISSTVLGKVIVQPDQNFT
<SEQ ID NO: 11; PRT; Homo sapiens> α-chain of MET
ECKEALAKSEMNVNMKYQLPNFTAETPIQNVILHEHHIFLGATNYIYVLNEEDLQKVAEY
KTGPVLEHPDCFPCQDCSSKANLSGGVWKDNINMALVVDTYYDDQLISCGSVNRGTCQRH
VFPHNHTADIQSEVHCIFSPQIEEPSQCPDCVVSALGAKVLSSVKDRFINFFVGNTINSS
YFPDHPLHSISVRRLKETKDGFMFLTDQSYIDVLPEFRDSYPIKYVHAFESNNFIYFLTV
QRETLDAQTFHTRIIRFCSINSGLHSYMEMPLECILTEKRKKR
<SEQ ID NO: 12; PRT; Homo sapiens> β-chain of MET
STKKEVFNILQAAYVSKPGAQLARQIGASLNDDILFGVFAQSKPDSAEPMDRSAMCAFPI
KYVNDFFNKIVNKNNVRCLQHFYGPNHEHCFNRILLRNSSGCEARRDEYRTEFTTALQRV
DLFMGQFSEVLLTSISTFIKGDLTIANLGTSEGRFMQVVVSRSGPSTPHVNFLLDSHPVS
PEVIVEHTLNQNGYTLVITGKKITKIPLNGLG
<SEQ ID NO: 13; PRT; Artificial Sequence> MET Dx Ab LC
EIQMTQSPASLSASVGETVTITCRASENIYSYLAWYQRKQGRSPQLLVYNAKPLAEGVPSRFSGS
GSGTQFSLKINSLQPEDFGTYYCQHHYGTPFTFGSGTRLEIKRADAAPTVSIFPPSSEQLTSGGA
SVVCFLNNFYPKDINVKWKIDGSERQNGVLNSWIDQDSKDSTYSMSSTLTLIKDEYERHNSYTCE
ATHKTSTSPIVKSFNRNEC
<SEQ ID NO: 14; PRT; Artificial Sequence> MET Dx Ab HC
EVQLQQSGTVLARPGASVKMSCKASGYSFTSYWMYWVKQRPGQGLEWIGGFHPRNSGTNYNQKFK
GKAKLTAVTSASTAYMELSSLTNEDSAVYYCTRGYYYDGSFTYWGQGTLVTVSAAKTTPPSVYPL
APGSAAQTNSMVTLGCLVKGYFPEPVTVTWNSGSLSSGVHTFPAVLQSDLYTLSSSVTVPSSTWP
SETVTCNVAHPASSTKVDKKIVPRDCGCKPCICTVPEVSSVFIFPPKPKDVLTITLTPKVTCVVV
DISKDDPEVQFSWFVDDVEVHTAQTQPREEQFNSTFRSVSELPIMHQDWLNGKEFKCRVNSAAFP
APIEKTISKTKGRPKAPQVYTIPPPKEQMAKDKVSLTCMITDFFPEDITVEWQWNGQPAENYKNT
QPIMDTDGSYFVYSKLNVQKSNWEAGNTFTCSVLHEGLHNHHTEKSLSHSPGK

1. A method of treating cancer, comprising administering to a patient with tumors bearing MET exon 14 skipping or MET exon 14 skipping phenotype an effective amount of merestinib, or a pharmaceutically acceptable salt thereof, in combination with an effective amount of emibetuzumab.

2. The method according to claim 1, wherein merestinib, or a pharmaceutically acceptable salt thereof, is administered at a dose of between about 60 mg to about 160 mg daily on a 28-day cycle and emibetuzumab is administered at a dose of about 500 mg to about 2000 mg on about day 1 and about day 15 of the 28-day cycle of merestinib dosing

3. The method according to claim 2, wherein merestinib, or a pharmaceutically acceptable salt thereof, is administered at a dose of between about 80 mg to about 120 mg daily on a 28-day cycle and emibetuzumab is administered at a dose of about 750 mg on about day 1 and about day 15 of the 28-day cycle of merestinib dosing.

4. The method according to claim 2, wherein merestinib, or a pharmaceutically acceptable salt thereof, is administered orally and emibetuzumab is administered intravenously.

5. The method according to claim 1, wherein the cancer is gastric cancer or lung cancer.

6. The method according to claim 1, wherein the cancer is lung cancer.

7. The method according to claim 6, wherein the cancer is non-small cell lung cancer.

8.-21. (canceled)
